(19) United States
(12) Patent Application Publication
(10) Pub. No.: US 2002/0182707 A1
(43) Pub. Date: Dec. 5, 2002

(54) Modified microbes

(76) Inventor: Richard D. Fuerle, Grand Island, NY (US)

Correspondence Address: RICHARD D. FUERLE, 1711 W. RIVER RD., GRAND ISLAND, NY, 14072, US

(21) Appl. No.: 10/064,738

(22) Filed: Aug. 12, 2002

Related U.S. Application Data
(63) Continuation-in-part of application No. 09/785,709, filed on Feb. 16, 2001, now Pat. No. 6,455,257.

(51) Int. Cl. C12N 1/21; C12N 15/00

(52) U.S. Cl. 435/252.3; 435/440

(57) ABSTRACT

Modified microbes that attack target cells but do not attack non-target cells are made from existing microbes by a process performed in a fluid that circulates in a loop. Microbes are presented with an opportunity to attack the target cells. Microbes that have a lesser affinity for the target cells are removed from the loop and microbes that have a greater affinity for the target cells are left within the loop. Microbes are presented with an opportunity to attack the non-target cells. Microbes that have a greater affinity for non-target cells are removed from the loop and microbes that have a lesser affinity for the non-target cells are left within the loop. Microbes within the loop are mutated.

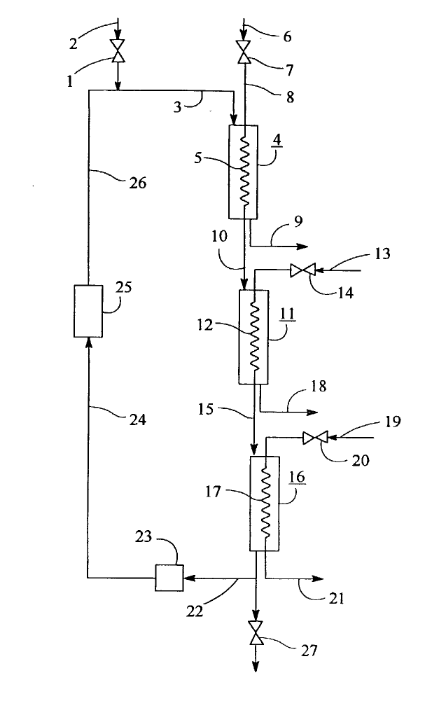

### CROSS REFERENCE TO RELATED APPLICATIONS

This application is a continuation-in-part of application Ser. No. 09/785,709, filed Feb. 16, 2001, now U.S. Patent No.

### BACKGROUND OF INVENTION

This invention relates to a microbe (a microorganism) made by modifying an existing microbe. In particular, it relates to a modified microbe that attacks target cells but does not attack non-target cells.

Many plants, animals, and bacteria are undesirable or even dangerous, yet are difficult to destroy without injuring other nearby life forms. This is particularly true when the undesirable life form is closely related to the desirable life form. In that case, a biocide that attacks the undesirable life form may also kill, or at least harm, the closely related desirable life form.

A designer disease is a man-made disease that attacks particular life forms (targets) while being relatively innocuous towards other life forms (non-targets).

### SUMMARY OF INVENTION

I have discovered modified microbes that can cause designer-diseases. In this invention, existing microbes are modified until they attack cells of a target population, but do not attack cells of a non-target population, even when the non-target population is closely related to the target population. The modified microbes of this invention can be made in apparatus that is inexpensive and simple to construct.

### BRIEF DESCRIPTION OF DRAWINGS

The accompanying drawing is a diagram illustrating a certain presently preferred embodiment of an apparatus suitable for producing modified microbes according to this invention.

### DETAILED DESCRIPTION

In the drawing, valve 1 is opened and the microbe to be modified is carried in fluid from line 2 through valve 1 into line 3 to the top of diffusor 4, which fills with microbe-containing fluid outside coil 5. Valve 1 is then closed. (After the initial charge of microbes, additional microbes should not be added to the system again.) Fluid, containing cells from the target population, enters line 6, passes through valve 7, then goes through line 8 into coil 5. Coil 5 is made of a semipermeable material. The microbes can pass through the semipermeable material but the target cells cannot. Some of the microbes in diffusor 4 pass from the outside of coil 5 to the inside of coil 5.

As the microbes move back and forth across the semipermeable material of coil 5, microbes that have a greater affinity for the target cells will be more likely to be inside coil 5 and microbes that have a lesser affinity for the target cells will be more likely to be outside coil 5. That is, target cells and non-target cells display on their sufaces particular molecules, such as glycoproteins. To a microbe, these displayed molecules appear as positive and negative charges of various magnitudes at particular positions in three-dimensional space. (The charges may also vibrate or resonate.) The microbes also have on their surfaces molecules they use to attach to a cell so that they can attack the cell. These molecules also constitute positive and negative charges of various magnitudes at particular positions in three-dimensional space. Different microbes, either initially or after they have been mutated, will have displays of charges that differ in their polarity (+ or −), magnitude, and/or coordinates. Those microbes that have surface molecules whose charges better interlock (i.e., positive charges on the microbe matched with negative charges on the cell and negative charges on the microbe matched with positive charges on the cell, at corresponding positions) will have a greater affinity for the target cells or the non-target cells. As the modification process proceeds, the microbes retained in the system will have greater and greater affinities for the target cells, and lesser and lesser affinities for the non-target cells, until they attack and destroy the target cells, but have little or no attraction towards the non-target cells. Microbes outside coil 5, which have less affinity for the target cells, are flushed out of diffusor 4 through line 9 as waste.

The mixture of target cells and microbes inside coil 5 passes though line 10 to diffusor 11 and fills diffusor 11 outside of coil 12, which is made of a semipermeable material. Fluid, without microbes or cells in it, is sent though line 13, pass valve 14, to the inside of coil 12. Microbes outside coil 12 pass to the inside of coil 12 and are sent through line 15 to diffusor 16, filling diffuser 16 outside of coil 17, which is made of a semipermeable material. Initially, the microbes may attach to the target cells without actually attacking and destroying them. In that case, the microbes should be dislodged from the target cells so that they can enter the inside of coil 12. This can be accomplished by, for example, vibration, ultrasound, the use of chemicals, or other methods. The mixture of target cells, debris, and microbes outside coil 12 is flushed through line 18 to waste.

Fluid, containing cells from the non-target population, is sent through line 19, pass valve 20, to the inside of coil 17, then to waste in line 21. Some of the microbes outside coil 17 pass to the inside of coil 17. Some of the microbes inside coil 17 may attack non-target cells inside coil 17. Microbes inside coil 17 that do not attack non-target cells generally do not adhere to or remain in close proximity to non-target cells and can pass back to the outside of coil 17 again. Microbes inside coil 17 are more likely to have a greater affinity for non-target cells and to attack, adhere to, or remain near the cells, and are flushed out as waste in line 21. Microbes outside coil 17 pass through line 22 to pump 23, which pumps the fluid through line 24 to mutation station 25 where some of the microbes in the fluid are mutated. The fluid leaves mutation station 25 and passes though line 26 to line 3, forming a loop.

Thus, diffusor 4 gives the microbes an opportunity to attack target cells. Those that do go to diffusor 11 along with the target cells or pieces of target cells. Diffusor 11 separates the microbes that attacked the target cells from the target cells and other debris. Those microbes then go to diffusor 16 which gives the microbes an opportunity to attack non-target cells. Those that do are discarded. The microbes that leave diffusor 16 are those more likely to attack target cells and/or not attack non-target cells. In mutation station 25, the microbes are mutated, hopefully creating mutations that are even better at attacking target cells and/or not attacking non-target cells. The microbes are circulated and mutated repeatedly until they display the desired degree of abilities to attack target cells and not attack non-target cells. They can then be removed and collected by opening valve 27. It is also contemplated that the positions of diffusers 11 and 16 can be reversed, so that microbes that attack non-target cells are removed before the remaining microbes are separated from target cells and debris.

It will be necessary to adjust valve 7 so that the flow of target cell-containing fluid is slow enough to give the microbes time to attack and then get free of the target cells, yet fast to keep the process moving. If viruses are used, for example, the residence time of the target cells in diffusors 4 and 11 should be long enough for the viruses to enter the cells, reproduce, burst out of the cells, and pass into coil 12 before the contents of diffuser 11 are discarded. Valve 20 should be open enough so that there are always non-target cells available to be attacked inside coil 17. Valves (not shown) can also be placed on lines 9, 18, and 21 to control flow rates. If possible, fluid flows in the diffusors should be laminar and turbulence should be minimized. The percentage (“α”) of the microbes in diffuser 4 that are discarded in line 9 for not attacking target cells and the percentage (“β”) of microbes in diffusor 16 that are removed through line 21 for attacking non-target cells are preferably high, about 90 to about 99.9%. (The percentage of microbes removed by diffusor 11 should be minimized.) Circulation time around the loop can be increased as the microbes evolve to forms that more rapidly attack the target cells. A sample of the microbes can be taken and stored periodically so that the experiment can be restarted using the last sample taken should a catastrophic failure occur. In order to prevent the creation of microbes that are inferior in their ability to attack target cells without attacking non-target cells, mutation of the microbes should cease once the microbes have these abilities to the extent desired. Circulation around the loop should then be continued for a while to weed out any microbes still present in the apparatus that attack non-target cells.

Diffusors 4, 11, and 16 are preferably made of transparent glass or plastic for observational purposes, but other materials, such as metal or ceramic, can also be used. Indeed, simple jars could suffice, though it is desirable that they be sealed to prevent other microbes from entering the system. Cylindrical diffusers are preferred, though other shapes can also be used. By placing diffusor 4 above diffusor 11 and diffusor 11 above diffusor 16, the fluid will flow under gravity so that it is possible to operate the apparatus using only a single pump 23.

Coils 5, 12, and 17 are made of a semipermeable material, that is, a material having openings therethrough that are large enough for the microbes to pass through but are too small for the cells to pass through. The coils are preferably made of a flexible polymeric membrane, but could also be made of porous ceramic, paper, or other suitable materials. An inexpensive coil could even be made by rolling up filter paper and sealing the edge with waterproof glue. To increase the transport of the microbes into and out of the coils, it is preferable to maximize the surface area of the coil and minimize the volume of fluid inside and outside the coils. While coils are preferred for their high surface area, other shapes can also be used.

The mutation station is preferably an ultraviolet light or a bank of ultraviolet lights as they are easy to use and effective. Mutation can also be accomplished by using chemical mutation agents, heat, radiation, or other means.

Target and non-target cells include plant cells, animal cells, and bacteria. Target cells will usually be selected from a population that is harmful to the non-target population. Examples of non-target and target populations include crops and weeds; birds and geckos on Guam and the brown tree snakes that prey on them; fish used for food and less desirable competing fish; humans and intestinal worms or amoebae; humans and drug-resistant bacteria; normal cells and cancer cells; etc. The invention is particularly useful when the target and non-target cells are from the same species as it is difficult for other biocides to distinguish between cells of the same species. The type of target and non-target cells used are preferably those most likely to be attacked by the microbe. For example, if the target population is streptococci, the non-target population is children, and the microbe is a bacteriophage, human epithelial cells may be a good choice for the non-target cells. Of particular interest are target cells that are cancerous and non-target cells that are non-cancerous normal cells. The normal cells preferably would include cells from a variety of different types of tissue, including cells from the same type of tissue as the cancerous cells. For example, if a patent had prostate cancer and the target cells are prostate cancer cells, the non-target cells would preferably include both normal prostate cells as well as other types of cells, such as muscle cells, liver cells, etc., to be sure that the modified microbe did not attack any cells in the body.

Cells from any type of cancer could be used for target cells, including prostate cancer, breast cancer, colon cancer, pancreatic cancer, lung cancer, and leukemia. A microbe can be modified so that it attacks the cancer cells (e.g., prostate cancer cells) but does not attack the corresponding normal cells (e.g., normal prostate cells) because cancer cells display abnormal molecules, such as glycoproteins, on their surface that are not displayed by normal cells.

There are many types of prostate cancer, many types of breast cancer, etc., each type of prostate cancer, breast cancer, etc., having a different DNA profile. For example, there may be 10 different types of prostate cancer, each type having a different sequence of bases (guanine (G), adenine (A), cytosine (C), and thymine (T)) one or more genes. Preferably, the target cancer cells should all have the same DNA profile, at least for those genes that code for molecules displayed on the surface of the cell. While the relevant genes for each type of prostate cancer can be sequenced, a surrogate for the relevant base sequences, such as a Gleason score, could be used. That is, if a prostate cancer patent had a Gleason score of 8, one could use as target cells prostate cancer cells taken from one or more patients whose Gleason score was 8. Thus, a modified microbe could be made that would attack each specific type of prostate cancer, breast cancer, etc.

In addition, since each person's sequence of DNA bases differs on at least some genes and those differences may cause slightly different molecules to be displayed on the surface of that patient's cancer cells, the modified microbe can, if desired, be customized for a particular patient. For example, if the patient had prostate cancer, the type of prostate cancer the patient had could be identified (e.g., Gleason score=8) and a modified microbe could be made (or selected, if already made) that would attack that type of prostate cancer (but not normal cells). That modified microbe could then be further modified to attack cancer cells that have the DNA profile of a particular patient, at least for those genes that code for surface molecules. Those cells could simply be taken from the patient in a biopsy or surgery or they could be made by genetic engineering.

The microbes are disease-causing agents that are smaller than the target cells and the non-target cells so that they can pass through the semipermeable material but cells from the target and non-target populations cannot. The microbes can be prions, viruses, prokaryotes such as bacteria, and even small protozoa. While one can begin with a microbe that neither attacks the target population nor is harmless to the non-target population, it is preferable to select a microbe that occurs naturally in the same environment as the target population and either already attacks the target population, or at least life forms similar to the target population, or already is harmless, or relatively harmless, to the non-target population. For example, to make a designer disease that will attack a weed, but not nearby crops, it would be preferable to select a microbe that already attacks the weed or a plant that is similar to the weed and/or is harmless or almost harmless to the crop. The simplest microbes (e.g., viruses) are preferred to the more complex microbes as they evolve faster. It is preferable to use a mixture of genetically dissimilar microbes as that increases the probability of evolving a microbe that attacks target cells but not non-target cells. For example, if a particular virus is to be used, it is preferable to use a mixture of various strains of that virus.

The microbe to be modified should be carefully selected, particularly if the modified microbe is to be injected into a human. While the modification process should discard any microbe that attacks non-target cells, it is possible for unmodified microbes to get stuck in some portion of the apparatus so that they are not discarded. Then, if the unmodified microbes become unstuck as the modified microbes are being removed from the apparatus, they could be removed with the modified microbes. To eliminate that possibility, one can avoid using dangerous microbes that the non-target population (e.g., humans) cannot easily destroy, such as HIV or smallpox (variola). Alternatively, if a dangerous microbe is the best candidate, the microbes removed from the apparatus can be injected into a new sterile apparatus and the process repeated for a while without mutating the microbes, thereby removing any remaining unmodified microbes.

If the target cells are cancer cells, the microbe is preferably an RNA or DNA virus. Preferred choices for viruses include the adenoviruses and rhinoviruses, which cause colds, as they are relatively harmless and the body can usually destroy them easily after the patient has been treated. In order for the modified virus to destroy all the cancer cells before the body destroys the virus, the virus is preferably injected directly into the cancer. If that is not possible (e.g., cancers such as leukemia, bone cancer, lung cancer, and metastasized cancers), the virus would be injected into the blood stream. If the body's immune system attacks the virus too quickly, it may be desirable to temporarily impair the immune system.

While the modified viruses will enter the cancer cells and direct the cells to make more viruses, which will then attack additional cancer cells, it is preferable not to rely on the reproduction of the virus within the body to kill all the cancer cells because the body may create antibodies to the virus before the viruses can reproduce. In addition, the viruses may evolve during reproduction so that they attack normal cells. For those reasons, it is preferably to give a one-shot treatment using far more virus particles than there are cancer cells, rather than a series of partial treatments.

The fluid used should be water-based and should not kill the target cells, the non-target cells, or the microbes. A saline solution can be used, for example, that preferably contains the nutrients needed by the target and non-target cells. The microbes, however, should feed on the target cells. If the microbes initially used are not able to feed on the target cells, it will be necessary to provide them with food until they can do so.

If the microorganism is a virus, it must reproduce by entering a cell and directing the cell to make more virus particles. If initially it cannot use target cells for that purpose, it will be necessary to supply the virus with other cells that it can use for that purpose. This can be accomplished in several ways. For example, if the desired target cells are prostate cancer cells but there is no suitable virus that attacks prostate cancer cells, one could select a virus that attacks epithelial cells, such as an adenovirus or a rhinovirus, and use mixture of epithelial cells and prostate cancer cells as target cells, gradually increasing the proportion of prostate cancer cells. Another method is to put the desired target cells in diffusor 4 and the cells the virus already attacks in diffusor 16. For example, if a rhinovirus is used that attacks epithelial cells but not prostate cancer cells, prostate cancer cells would be placed in diffusor 4 as the target cells and epithelial cells would be placed in diffusor 16 as the non-target cells. Initially, the percentage α for diffusor 4 and the percentage β for diffusor 16 would be set so that the diffusors discard only enough virus particles to keep the number of virus particles in the system about constant. As the virus is modified so that it can enter and reproduce in the prostate cancer cells in diffusor 4, the number of virus particles in the system will increase and the cells in diffusor 16 can be changed to normal prostate cells.

1. A modified microbe made by a process that comprises circulating a fluid in a loop and within said fluid
(A) presenting microbes with an opportunity to attack target cells;
(B) removing from said loop microbes having a lesser affinity for said target cells and leaving within said loop microbes having a greater affinity for said target cells;
(C) presenting microbes with an opportunity to attack non-target cells;
(D) removing from said loop microbes having a greater affinity for said non-target cells and leaving within said loop microbes having a lesser affinity for said non-target cells; and
(E) mutating microbes within said loop.

2. A modified microbe according to claim 1 wherein microbes are removed in step (B) by providing a semipermeable membrane through which said microbes can pass but said target cells cannot pass, where said membrane separates said fluid into a first part containing said microbes and a second part containing said microbes and said target cells, and removing said first part from said loop.

3. A modified microbe according to claim 1 wherein microbes are removed in step (D) by providing a semipermeable membrane through which said microbes can pass but said non-target cells cannot pass, where said membrane separates said fluid into a first part containing said microbes and said non-target cells and a second part containing said microbes, and removing said first part from said loop.

4. A modified microbe according to claim 1 wherein said target cells are from the same species as said non-target cells.

5. A modified microbe according to claim 1 wherein said target cells and said non-target cells are from different species.

6. A modified microbe according to claim 1 wherein said non-target cells are human.

7. A modified microbe according to claim 1 wherein said microbes are bacteria.

8. A modified microbe according to claim 1 wherein said microbes are viruses.

9. A modified microbe according to claim 8 wherein said target cells are cancer cells and said non-target cells are normal cells.

10. A modified microbe according to claim 9 wherein said viruses are selected from the group consisting of adenoviruses, rhinoviruses, and mixtures thereof.

11. A modified microbe according to claim 1 wherein ultraviolet light is used to mutate said microbes.

12. A modified microbe made in an apparatus that comprises
(A) first, second, and third containers, each having two sections, I and II, separated by a semipermeable material through which a microbe can pass but cells from target and non-target populations cannot pass, and an entrance and an exit to each section, where the entrance to section I of said first and second containers is a circulation entrance, the exit from section II of said first and second containers is a circulation exit, and the entrance to and exit from section I of said third container is a circulation entrance and a circulation exit, respectively;
(B) conduits forming a loop by connecting the circulation exit of each container to the circulation entrance of another container;
(C) a pump for moving fluid around said loop; and
(D) means for mutating microbes in said fluid;
by a process that comprises
(A) passing cells from a target population in an aqueous fluid into the entrance to section II of said first container;
(B) passing an aqueous fluid without microbes or cells in it into the entrance of section II of said second container;
(C) passing cells from a non-target population in an aqueous fluid into the entrance of section II of said third container;
(D) adding microbes to said loop;
(E) circulating said aqueous fluid around said loop;
(F) mutating some of the microbes in said loop; and
(G) collecting microbes from said loop after they have an enhanced ability to attack cells from said target population or to not attack cells from said non-target population.

13. A modified microbe according to claim 12 wherein sections I are cylinders and sections II are tubes within said cylinders, and said tubes are made of said semipermeable material.

14. A modified microbe according to claim 12 wherein one of said containers is higher than the other two containers and one of the two lower containers is higher than the other of the two lower containers, so that said fluid flows by gravity from the highest container to the lowest container.

15. A modified virus that attacks cancer cells but does not attack normal cells, made by a process that comprises circulating a fluid in a loop and within said fluid
(A) forming a first mixture of said cancer cells and particles of at least one type of virus;
(B) removing from said loop virus particles having a lesser affinity for said cancer cells and leaving within said loop virus particles having a greater affinity for said cancer cells;
(C) forming a second mixture of said normal cells and said virus particles;
(D) removing from said loop virus particles having a greater affinity for said normal cells and leaving within said loop virus particles having a lesser affinity for said normal cells; and
(E) mutating virus particles within said loop.

16. A modified virus according to claim 15 wherein said cancer cells are prostate cancer cells.

17. A modified virus according to claim 15 wherein said cancer cells are breast cancer cells.

18. A modified virus according to claim 15 wherein said normal cells include normal cells from the same type of tissue as said cancer cells.

19. A modified virus according to claim 15 wherein said cancer cells all have the same DNA profile for genes that code for molecules on their surface.

20. A modified microbe according to claim 15 wherein said viruses are selected from the group consisting of adenoviruses, rhinoviruses, and mixtures thereof.
